(19) United States
(12) Patent Application Publication
(10) Pub. No.: US 2023/0248791 A1
(43) Pub. Date: Aug. 10, 2023

(54) METHODS FOR MANUFACTURING ANGELICA GIGAS NAKAI EXTRACT USING FERMENTATION BACTERIA

(71) Applicant: Sooy-K Co., Ltd., Seongnam-si (KR)

(72) Inventors: Youn Ok JUNG, Namwon-si (KR); Ji Sun CHUNG, Seongnam-si (KR); Kyoung Mo CHUNG, Seoul (KR)

(21) Appl. No.: 17/866,911

(22) Filed: Jul. 18, 2022

(30) Foreign Application Priority Data
Feb. 7, 2022 (KR) 10-2022-0015345

(51) Int. Cl. A61K 36/232; A61K 35/742; C12P 1/04; C12N 1/20; A61K 31/37

(52) U.S. Cl. CPC A61K 36/232; A61K 35/742; C12P 1/04; C12N 1/205; A61K 31/37; C12R 2001/125

(57) ABSTRACT

Proposed is a method for manufacturing an Angelica gigas Nakai extract with an increased decursin content, an Angelica gigas Nakai extract prepared by the method, and a cosmetic composition including the Angelica gigas Nakai extract as an active ingredient, the manufacturing method including the steps of extracting Angelica gigas Nakai with ethanol; and inoculating a strain of the genus Bacillus into the ethanol extract of Angelica gigas Nakai to induce fermentation, the strain of the genus Bacillus being selected from Bacillus amyloliquefaciens and Bacillus subtilis.

### BACKGROUND OF THE INVENTION

### (A) Field of the Invention

The present invention relates to a method for manufacturing an Angelica gigas Nakai extract using fermentation bacteria, and particularly to a method for manufacturing an Angelica gigas Nakai extract with an increased decursin content, an Angelica gigas Nakai extract manufactured by the method, and a cosmetic composition including the Angelica gigas Nakai extract, the manufacturing method including the steps of extracting Angelica gigas Nakai with ethanol; and inoculating a strain of the genus Bacillus into the ethanol extract of Angelica gigas Nakai to induce fermentation, the strain of the genus Bacillus being selected from Bacillus amyloliquefaciens and Bacillus subtilis.

### (B) Description of the Related Art

Natural medicines refer to plants, animals, minerals and microorganisms obtained from nature and their metabolites, and are also called crude drugs or herbal medicines. Research studies on natural medicines have been steadily conducted, but in particular, with a recent increase in the preference for eco-friendly and naturally derived ingredients, the natural medicines have become in the spotlight.

Dang-Gui, in Korean medicine, refers to the dried roots of Angelica gigas Nakai belonging to the umbelliferae/parsley family and has been used as a representative herbal medicine for hemostatic agents in East Asia since ancient times. As stated in Dong-Ui medicine, Dang-Gui (Angelica root) offers three health benefits: tonifying the blood and regulating the menses (Bohyeoljogyeong), promoting blood circulation to alleviate pain (Hwanlhyeoljitong), and supporting lubrication of the intestines (Yoonjang). Angelica root is not only used as a treatment for high blood pressure, anemia and extravasated blood but also as a sedative, a painkiller, or a tonic medicine. Particularly, it is widely used for gynecological diseases due to its beneficial effects of regulating the menses and strengthening the uterus before and after childbirth.

Angelica gigas Nakai contains several types of coumarins, including decursin, decursinol angelate, nodakenin, and isoimperatonin, essential fats, polyacetylene, etc. The main active ingredients are pyranocoumarin-based components, that is, decursin and decursinol angelate. Accordingly, the pharmacological effects of Angelica gigas Nakai used for the treatment or prevention of various diseases have been steadily reported in relation to these ingredients.

Decursin (3-methyl-2-butenoic acid 2,2-dimethyl-8-oxo-3,4-dihydro-2H,8H-pyrano[3,2-g]chromen-3-yl ester) has a chemical formula as given below. It has been reported that Decursin has a variety of biological efficacies, such as supporting anti-tumor and/or antibacterial activities, improving blood circulation and/or diabetes, inhibiting metabolic enzymes, offering anti-inflammatory and antioxidant activities, helping tissue regeneration, and promoting cognitive functions. Decursin is notably attracting attention as an active ingredient in cosmetics. It protects the skin from free radicals due to its excellent antioxidant effects to prevent skin aging and wrinkles, promotes blood circulation to facilitate the supply of nutrients to the skin, and exhibits anti-inflammatory, antibacterial and skin pigmentation inhibitory effects.

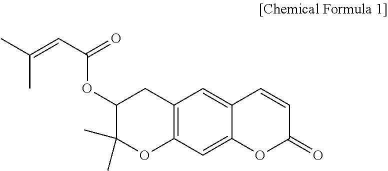 [SMILES: CC(C)=CC(=O)OC1Cc2cc3ccc(=O)oc3cc2OC1(C)C]

In association with these efficacies, research studies have been actively conducted to manufacture extracts or fractions having a high content of useful bioactive ingredients such as decursin from Angelica gigas Nakai. For example, Korean Patent Registered Publication No. 10-1898387 discloses a method for preparing a polysaccharide extract of Angelica gigas Nakai that includes: extracting Angelica gigas Nakai with hot alcohol to obtain an alcohol extract of Angelica gigas Nakai; extracting the alcohol extract of Angelica gigas Nakai with hot water and producing dry powder; conducting extraction of the dry powder with alcohol under agitation and then filtration to obtain a solid; and drying the solid to obtain a powdered polysaccharide extract of Angelica gigas Nakai. Korean Patent Laid-Open Publication No. 10-2018-0079967 states a preparation method for decursinol that includes: conducting long-term reflux of Angelica gigas Nakai powder in ethyl acetate and then condensation/filtration; ethylacetate-washing/concentrating the filtrate; condensing with added methanol; agitating with added sodium hydroxide; condensing with added methylene chloride and water; treating with acids; isolating a methylene chloride layer; and conducting recrystallization under agitation in a mixed solution of methanol and t-butyl methyl ether at volume ratio of 1:2. Korean Patent Registered Publication No. 10-1819450 discloses a purification method for an extract solution of Angelica gigas Nakai that includes: cold-brewing Angelica gigas Nakai in ethanol and conducting hot extraction; removing a precipitate from the isolated filtrate; isolating and concentrating the supernatant; and isolating the extract solution of Angelica gigas Nakai with acidified alcohol on a column chromatograph. Korean Patent Laid-Open Publication No. 10-2017-0059197 describes a method for preparing an extract concentrate of Angelica gigas Nakai that includes: extracting Angelica gigas Nakai with water under oxygen-free high-temperature and high-pressure conditions and conducting filtration to obtain a solid; extracting the solid with ethanol; and concentrating the resultant extract. Korean Patent Laid-Open Publication No. 10-2010-0060709 discloses a preparation method for decursin that includes: obtaining an Angelica gigas Nakai extract by supercritical extraction; and isolating and purifying decursin on a column chromatograph. Korean Patent Registered Publication No. 10-1056352 specifies an extraction method for Angelica gigas Nakai that involves applying a high-voltage pulse electric field to the hot-water extract of Angelica gigas Nakai. Korean Patent Registered Publication No. 10-0749233 describes a method for preparing an Angelica gigas Nakai extract, characterized by extracting Angelica gigas Nakai with hot ethanol in the presence of an alkalifying agent. In addition, Korean Patent Registered Publication No. 10-0775741 discloses a method for preparing an Angelica gigas Nakai extract that includes extracting dry Angelica gigas Nakai with hot ethanol; concentrating the filtrate obtained from the hot-ethanol extraction under vacuum; re-solubilizing the concentrate in hot ethanol; standing the re-solubilized concentrate at low temperature to form carbohydrate crystals; conducting filtration with a filter; and concentrating the filtrate under vacuum.

These various conventional methods for preparing extracts or fractions containing decursin or the like of Angelica gigas Nakai are problematic in that their processes and necessary facilities are too complicated. Accordingly, the inventors of the present invention have tried to find a preparation method that increases the decursin content in the Angelica gigas Nakai extract while involving less complexity of the process and facility, and discovered that the decursin content in the extract can be raised by inoculating a strain of the genus Bacillus, especially Bacillus amyloliquefaciens EMD17 or Bacillus subtilis 9-3, into the ethanol extract of Angelica gigas Nakai to induce fermentation, thereby completing the present invention.

### SUMMARY OF THE INVENTION

### Technical Problem

For solving the problems with the prior art, it is an object of the present invention to provide a method for increasing the content of active ingredients, especially decursin, in the Angelica gigas Nakai extract, an Angelica gigas Nakai extract prepared by the manufacturing method, and a cosmetic composition including the Angelica gigas Nakai extract.

The above object of the present invention is not intended as a definition of the limits of the invention. The above and other objects of the invention will become apparent to those skilled in the art from the following description of embodiments.

### Technical Solution

In order to achieve the object of the present invention, one aspect of the present invention is directed to a method for manufacturing an Angelica gigas Nakai extract with an increased decursin content, characterized by: extracting Angelica gigas Nakai with ethanol; and inoculating a strain of the genus Bacillus into the ethanol extract to induce fermentation, the strain of the genus Bacillus being selected from Bacillus amyloliquefaciens and Bacillus subtilis.

In an embodiment of the present invention, the Angelica gigas Nakai is dried.

In an embodiment of the present invention, the Angelica gigas Nakai is the root part of Angelica gigas Nakai.

In an embodiment of the present invention, the ethanol is an ethanol containing 30 to 70% ethanol by volume.

In an embodiment of the present invention, after the extraction step, the ethanol extract is powdered and subjected to inoculation of the strain of the genus Bacillus.

In an embodiment of the present invention, the strain of the genus Bacillus is selected from Bacillus amyloliquefaciens EMD17 and Bacillus subtilis 9-3.

In an embodiment of the present invention, the fermentation is conducted for 5 to 7 days.

In an embodiment of the present invention, the fermentation is conducted at 25 to 35° C.

Another aspect of the present invention is directed to an Angelica gigas Nakai extract prepared by the above-described manufacturing method.

Further another aspect of the present invention is directed to a cosmetic composition comprising the above-described Angelica gigas Nakai extract as an active ingredient.

The foregoing technical solutions are to be construed as merely illustrative, and not limitative of the present invention. In addition to the exemplary embodiments described above, additional embodiments may exist in the drawings and detailed description of the present invention.

### Effects of Invention

The method for manufacturing an Angelica gigas Nakai extract according to the present invention can produce an Angelica gigas Nakai extract with an increased decursin content through a simple process. This allows it to prepare an Angelica gigas Nakai extract and a cosmetic composition containing the Angelica gigas Nakai extract that have potential benefits, such as preventing or improving skin photoaging, wrinkles or pigmentation and providing antibacterial, anti-inflammatory effects.

The effects of the present invention are not limited to the above effects, but should be construed to include all effects that can be inferred from the configuration of the invention disclosed in the description or claims of the present invention.

### BRIEF DESCRIPTION OF DRAWINGS

FIG. 1 presents the analytical results for the ingredients of an Angelica gigas Nakai extract.

FIG. 2 presents the analytical results for the ingredients of an extract obtained 2 days, 4 days, and 6 days after inoculation of Bacillus subtilis 9-3 strains into an ethanol extract of Angelica gigas Nakai.

FIG. 3 presents the analytical results for the ingredients of an extract obtained 6 days after inoculation of different strains of the genus Bacillus into an ethanol extract of Angelica gigas Nakai.

### DETAILED DESCRIPTION OF THE EMBODIMENTS

The method for manufacturing an Angelica gigas Nakai extract with an increased decursin content in accordance with one aspect of the present invention is characterized by including: extracting Angelica gigas Nakai with ethanol; and inoculating a strain of the genus Bacillus into the ethanol extract of Angelica gigas Nakai to induce fermentation, the strain of the genus Bacillus being selected from Bacillus amyloliquefaciens and Bacillus subtilis.

The term “extract” as used herein refers to a material extracted from a raw material by any method, and includes, without limitation, an extract solution obtained thereby, a concentrate obstainable therefrom, and a dried or powdered form of the concentrate.

The extract may be obtained by extraction from a raw material or a dried form thereof. The raw material of the extract may include, without limitation, cultivated or commercially available ones.

In extracting a raw material to obtain the extract, the extraction method used in the present invention may include any known conventional extraction method, such as solvent extraction, ultrasonic extraction, high-pressure extraction, ultra-high pressure extraction, filtration extraction, or reflux extraction. Preferably, the extract may be prepared by using solvent extraction or reflux extraction. The extraction process may be repeated several times, and thereafter, an additional step of concentration or freeze-drying may be conducted. Specifically, the obtained extract may be concentrated under reduced pressure to form a concentrate, and the concentrate may be freeze-dried and powdered with a grinder into a high-concentration extract powder. The extract of the present invention includes a fraction obtained by additional fractionation of the extract or a fermented product obtained by an additional process such as fermentation of the extract.

Angelica is a perennial herbaceous plant belonging to the Umbrella family and is mainly cultivated for medicinal purposes in Korea, Japan, and China. Angelica species are classified according to their place of production: Angelica gigas Nakai produced in Korea, Angelica acutiloba Kitagaw in Japan, and Angelica sinensis Diels in China. The Angelica species are known to come in different ingredients and pharmacological effects. Among other Angelica species, Angelica gigas Nakai used in the present invention is well known to contain various active ingredients described above, especially decursin and decursinol angelate as main active ingredients.

In an embodiment of the present invention, the Angelica gigas Nakai may be dried. According to a research study on the fermentation of Angelica gigas Nakai with Lactobacillus, the decursin content is increased in the dried form of the Angelica gigas Nakai (Seo S H. (2016), Enhancement of Indicator Substances and Antioxidant Activities of Angelica gigas Nakai by Bioconversion Technology, Master's thesis, Andong University, Andong-si, South Korea).

In an embodiment of the present invention, the Angelica gigas Nakai may be any part of Angelica gigas Nakai selected from roots, leaves and stems. Preferably, the Angelica gigas Nakai may be a root part of Angelica gigas Nakai. According to a research study on the bioactive substances contained in each part of Angelica gigas Nakai, the decursin content in the leaves, stems and roots was 13.0%, 37.6% and 47.1%, respectively, revealing that the roots had the highest decursin content (Heo et al., Journal of Life Science, 2010, 20 (5): pp. 750-759).

In an embodiment of the present invention, the ethanol may be an ethanol containing 30 to 70% ethanol by volume, preferably 40 to 60% ethanol by volume.

In an embodiment of the present invention, after the extraction step, the ethanol extract may be powdered and subjected to inoculation of the strain of the genus Bacillus.

In an embodiment of the present invention, the strain of the genus Bacillus may be selected from Bacillus amyloliquefaciens EMD17 and Bacillus subtilis 9-3. EMD17 is a strain isolated from one of the Korean traditional fermented foods, Cheonggukjang, and has been identified by Lee, Jae-Yong (2015): Characteristics of antimicrobial substances produced by Bacillus amyloliquefaciens EMD17 (Master's thesis, Gyeongsang National University, Jinju-si, South Korea). Bacillus subtilis 9-3 is a strain deposited with the Korea Microorganism Conservation Center under accession number KCCM 11316 or a strain derived therefrom.

The term “fermentation” as used herein means bioconversion that converts organic matters into other compounds by enzymatic activities and can be achieved through inoculation of the above-mentioned strains.

In an embodiment of the present invention, the fermentation may be preferably conducted for 5 to 7 days. The researchers of the present invention have discovered that the extract exhibits different patterns of increase or decrease in the content of active ingredients depending on the period of the fermentation and that the decursin content significantly increases 6 days after inoculation of the strain of the genus Bacillus, particularly Bacillus amyloliquefaciens EMD17 or Bacillus subtilis 9-3. Preferably, the period of the fermentation may not exceed 7 days because a foul odor occurs after 7 days of the fermentation.

In an embodiment of the present invention, the fermentation may be conducted at 25 to 35° C., preferably 27 to 33° C.

In another aspect of the present invention, there is provided an Angelica gigas Nakai extract prepared by the above-described manufacturing method. The Angelica gigas Nakai extract has an increase in the content of decursin among other active ingredients of Angelica gigas Nakai.

In still another aspect of the present invention, there is provided a cosmetic composition comprising the Angelica gigas Nakai extract as an active ingredient.

According to a doctoral dissertation by Miae Yoo (2011) (Studies on regulation of skin anti-aging related proteins in Angelica gigas Nakai extracts and decursin-stimulated human dermal fibroblast, Ajou University, Suwon-si, South Korea), the Angelica gigas Nakai extract promotes the expression of proteins involved in collagen synthesis and extracellular matrix-fibroblast interaction, both of which play an important role in the suppression of skin aging, and such an effect increases in a manner dependent on the concentration of decursin in the extract. As stated in the doctoral dissertation, decursin inhibits UVA/UVB-induced skin damage and skin aging, and according to a clinical evaluation, decursin-containing functional cosmetics have been proved to offer beneficial effects to improve skin wrinkles, pigmentation and skin tone.

Choi et al. (Biomed Pharmacother, 2022, 147:112651) has reported that decursin regulates various signal transduction systems, such as PKA/CREB, p38/ERK MARK, and PI3K/Akt/GSK-3β to inhibit microphthalmia-associated transcription factors (MITFs), a major transcription factor in melanin synthesis, and prevents melanin synthesis in an ex vivo 3D human skin model, thereby specifically revealing the mechanisms of the skin whitening effect of decursin.

It is reported that decursin exhibits a strong antibacterial action (Lee et al., Arch Pharm Res, 2003, 26 (6): 449-452). Decursin is also known to have an anti-inflammatory effect through a mechanism of inhibiting cyclooxygenase-2 (COX-2) that is induced by internal/external stimuli to cause inflammation (Shehzad et al., Inflamm Res, 2018, 67 (3): 209-218, et.).

The composition of the present invention uses the Angelica gigas Nakai extract with an increased decursin content as an active ingredient to offer benefits of preventing or improving skin aging/photoaging, wrinkles or pigmentation; and providing an antibacterial or anti-inflammatory effect.

The cosmetic composition of the present invention may contain 0.01 to 50% by volume, preferably 0.1 to 10% by volume of the Angelica gigas Nakai extract.

As for active ingredients, the cosmetic composition of the present invention may include ingredients commonly used in the conventional cosmetic compositions in addition to the Angelica gigas Nakai extract. The commonly used ingredients include conventional adjuvants, such as antioxidants, stabilizers, solubilizers, vitamins, pigments, and fragrances; and carriers.

The cosmetic composition of the present invention may be made as any type of formulation conventionally applied in the related art, examples of which formulation may include, but is not limited to, solutions, suspensions, emulsions, pastes, gels, creams, lotions, powders, soaps, surfactant-containing cleansers, oils, powder foundations, emulsion foundations, wax foundations, and sprays.

More specifically, it may be prepared in the form of toners, nourishing toners, nourishing creams, massage creams, essences, eye creams, cleansing creams, cleansing foams, cleansing waters, packs, sprays, or powders.

The carrier components available in the present invention formulated as a paste, cream or gel may include animal oils, vegetable oils, waxes, paraffins, starch, tragacanth, cellulose derivatives, polyethylene glycol, silicone, bentonite, silica, talc, zinc oxide, etc.

The carrier components available in the present invention formulated as a powder or spray may include lactose, talc, silica, aluminum hydroxide, calcium silicate, polyamide powder, etc. Particularly, the present invention in a spray formulation may further include a propellant, e.g., chlorofluorohydrocarbon, propane/butane, or dimethyl ether.

The carrier components available in the present invention prepared as a solution or emulsion formulation may include a solvent, solubilizer, or emulsifier, e.g., water, ethanol, isopropanol, ethyl carbonate, ethyl acetate, benzyl alcohol, benzyl benzoate, propylene glycol, 1,3-butylglycol oil, glycerol fatty acid ester, polyethylene glycol, or sorbitan fatty acid ester.

The carrier components available in the present invention formulated as a suspension may include a liquid diluent, e.g., water, ethanol or propylene glycol; a suspending agent, e.g., ethoxylated isostearyl alcohol, polyoxyethylene sorbitol ester, or polyoxyethylene sorbitan ester; microcrystalline cellulose; aluminum metahydroxide; bentonite; agar; or tragacanth.

The carrier components available in the present invention formulated as a surfactant-containing cleanser may include aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkylamidobetaine, aliphatic alcohols, fatty acid glyceride, fatty acid diethanol amide, vegetable oils, lanolin derivatives, ethoxylated glycerol fatty acid ester, etc.

The present invention prepared as a soap, surfactant-containing cleanser, or surfactant-free cleanser formulation can be applied to the skin and then wiped off, removed, or washed off with water. Without specific limitation, for example, the soap formulation may be liquid soaps, powder soaps, solid soaps, or oil soaps; the surfactant-containing cleanser formulation may be cleansing foams, cleansing water, cleansing wipes, or cleansing packs; and the surfactant-free cleanser formulation may be cleansing creams, a cleansing lotions, cleaning water, or cleansing gels, but not limited thereto.

Hereinafter, the disclosure of the present invention will be described in further detail with reference to examples, which are given for the understanding of the disclosure of the present invention and not intended to limit the scope of the present invention.

### Example 1. Preparation of Angelica gigas Nakai Extract

Angelica gigas Nakai was cultivated at a farm located in Sancheong-gun, Gyeongnam for 3 years, harvested in the fall, and cut up. 200 g of dried Angelica gigas Nakai root was powdered and put in 1 L of 50% (v/v) ethanol for extraction. The resultant ethanol extract was concentrated, freeze-dried, and powdered. Then, pre-cultured strains in MRS medium were mixed in an amount of 5% (w/w) of the freeze-dried powder of the ethanol extract to induce solid-state culture under conditions of 30° C. and 45% humidity.

The strains used herein were Bacillus amyloliquefaciens EMD17, Bacillus amyloliquefaciens HCD2, Bacillus subtilis 9-3, Bacillus subtilis #8, and Bacillus subtilis 191. The pre-culture process was performed in a medium containing glucose 10%, peptone 5%, KNO₃ 2%, NH₄H₂PO₄ 2%, MgSO₄.7H₂O 0.5%, and CaCl₂ 0.1% in a shaking incubator (JEIOTECH, ISS-4075R, Seoul, South Korea) at 30° C. and 150 rpm for 72 hours.

In this regard, fermentation was carried out for no longer than 6 days because a very nasty smell might occur in 7 days of inoculation with two of those stains.

### Examples 2. Analysis on Angelica gigas Nakai Extract

Acquity UPIC (Waters, waters, USA) was used as an analytical instrument of which the column was a Waters Acquity BEH C18 column (2.1×100 mm, 1.7 ¥ìm) and solvent A and solvent B were water for HPLC (0.1% formic acid) and acetonitrile, respectively. The analysis was performed under gradient conditions. As for analysis conditions, the analysis was a solvent gradient analysis that involved a column oven temperature of 25° C., a mobile phase having a velocity of 0.4 ml/min, and a DAD analysis wavelength of 330 nm. The ferments were analyzed with UPLC-QTOF-Mass for quantitative/qualitative analysis focusing on indicators. For analysis conditions, the analysis was performed using Waters UPLC-QTOF-Mass of which the column was an ACQUITY BEH C18 chromatography column (2.1×100 mm, 1.7 um) with a column temperature of 35° C. and mobile phases A and B were water (0.1% formic acid) and acetonitrile, respectively. Besides, a mass spectrometry analysis was carried out for determination of molecular weights. In this regard, the analysis was performed focusing on nodakenin, decursin, decursinol, and decursinol angelate, which are known as indicators of Angelica gigas Nakai.

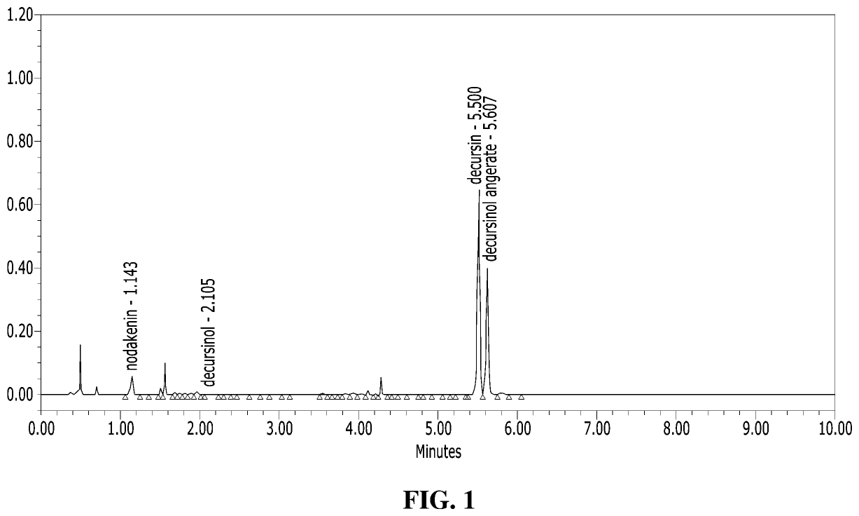

In the first place, the freeze-dried powder of the ethanol extract of Angelica gigas Nakai was analyzed to determine the contents of the active ingredients of Angelica gigas Nakai before fermentation. As a result, RT data showed nodakenin 1.143 ppm, decursinol 2.105 ppm, decursin 5.500 ppm, and decursinol angelate 5.607 ppm. The contents of the active ingredients were given as decursinol angelate 186.040 ppm>decursin 179.133 ppm>nodakenin 30.550 ppm>decursinol 3.615 ppm, indicating that decursinol angelate and decursin were present in highest amounts (FIG. 1).

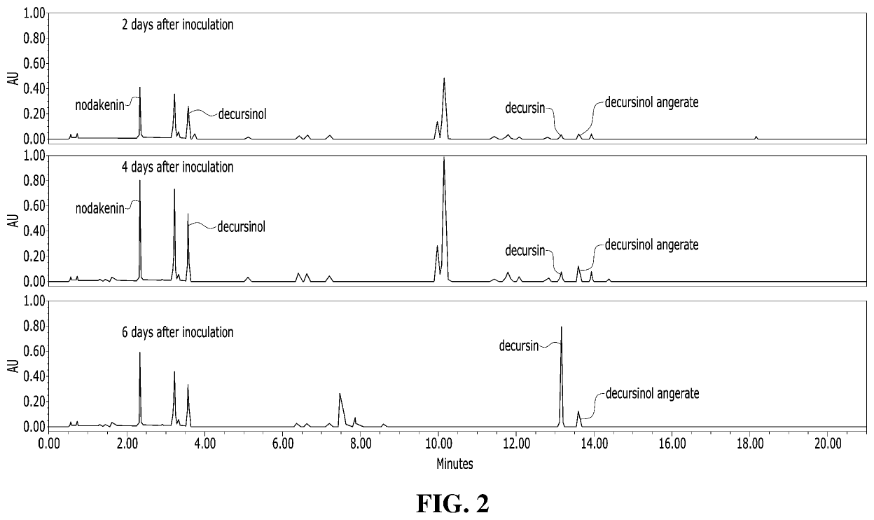

In the second place, Bacillus subtilis 9-3 was inoculated into the freeze-dried powder of the ethanol extract of Angelica gigas Nakai. Each Angelica gigas Nakai extract obtained 2, 4 and 6 days after inoculation was analyzed. The analytical results revealed, as shown in FIG. 2, that the contents of decursin and decursinol angelate plunged 2 days after inoculation, increased slightly 4 days after inoculation, and rose rapidly 6 days after inoculation. In contrast, the contents of nodakenin and decursinol increased 4 days after inoculation and decreased 6 days after inoculation.

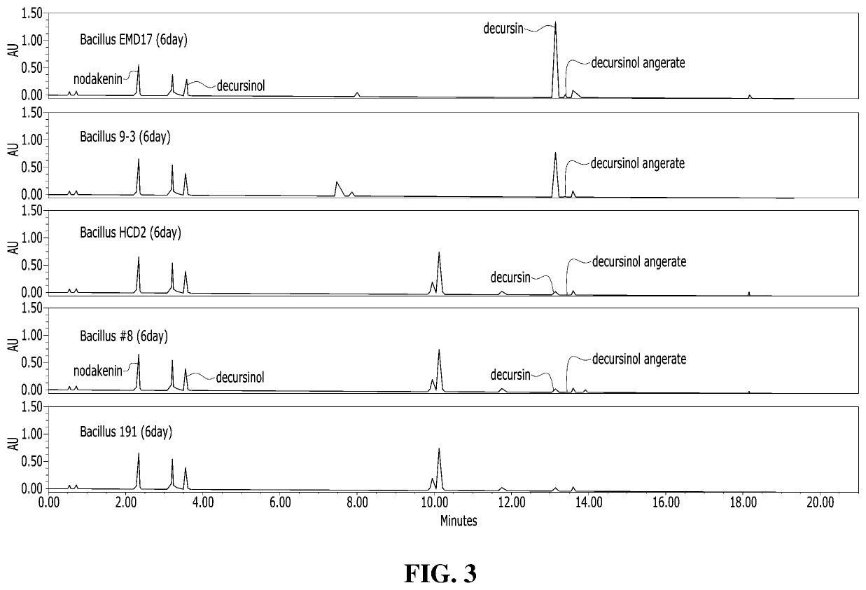

In the third place, different strains of the genus Bacillus were inoculated into the freeze-dried powder of the ethanol extract of Angelica gigas Nakai. Each Angelica gigas Nakai extract obtained 6 days after inoculation was analyzed. The analytical results confirmed, as shown in FIG. 3, that there was a big difference in the decursin content of the extracts for each strain. The Angelica gigas Nakai extracts obtained after inoculation of Bacillus amyloliquefaciens EMD17 or Bacillus subtilis 9-3 showed a high content of decursin, whereas those obtained after inoculation of Bacillus amyloliquefaciens HCD2, Bacillus subtilis #8 or Bacillus subtilis 191 contained only a trace amount of decursin. In contrast, the contents of nodakenin and decursinol were of similar values for all strains.

Taken together, the contents of active ingredients in the ethanol extract of Angelica gigas Nakai can be changed through the process of fermentation induced by inoculation of strains of the genus Bacillus, and those of specific ingredients can be varied depending on the type of the fermentation strain and the period of fermentation. The inventors of the present invention have confirmed that it is possible to increase the content of decursin through 6 days of fermentation induced by inoculation of a strain of the genus Bacillus, especially Bacillus amyloliquefaciens EMD17 or Bacillus subtilis 9-3.

The foregoing description of the present invention has been presented for purposes of illustration only. It should be apparent to those skilled in the present invention that many modifications and variations are possible without departing from the concept or essential features of the present invention. Therefore, the foregoing examples are to be construed as merely illustrative, and not limitative of the present invention. For example, each component described in singular form may be implemented in a scattered form, and likewise components described as scattered may be implemented in a combined form.

The scope of the present invention is defined by the appended claims and should be construed as including all changes or modifications derived from the meaning and scope of the claims and their equivalents.

What is claimed is:

1. A method for manufacturing an Angelica gigas Nakai extract with an increased decursin content, comprising:
extracting Angelica gigas Nakai with ethanol; and
inoculating a strain of the genus Bacillus into the ethanol extract of Angelica gigas Nakai to induce fermentation,
the strain of the genus Bacillus being selected from Bacillus amyloliquefaciens and Bacillus subtilis.

2. The method of claim 1, wherein the Angelica gigas Nakai is dried.

3. The method of claim 1, wherein the Angelica gigas Nakai is the root part of Angelica gigas Nakai.

4. The method of claim 1, wherein the ethanol is an ethanol containing 30 to 70% ethanol by volume.

5. The method of claim 1, wherein after the extraction step, the ethanol extract is powdered and subjected to inoculation of the strain of the genus Bacillus.

6. The method of claim 1, wherein the strain of the genus Bacillus is selected from Bacillus amyloliquefaciens EMD17 and Bacillus subtilis 9-3.

7. The method of claim 1, wherein the fermentation is conducted for 5 to 7 days.

8. The method of claim 1, wherein the fermentation is conducted at 25 to 35° C.

9. An Angelica gigas Nakai extract prepared by the manufacturing method according to claim 1.

10. A cosmetic composition comprising the Angelica gigas Nakai extract according to claim 9 as an active ingredient.
